(19) United States
(12) Patent Application Publication
(10) Pub. No.: US 2026/0355748 A1
(43) Pub. Date: Nov. 12, 2026

(54) BIOSENSORS MADE BY ADDITIVE MANUFACTURING

(71) Applicant: BOARD OF REGENTS, THE UNIVERSITY OF TEXAS SYSTEM, Austin, TX (US)

(72) Inventors: Mohammed MANIRUZZAMAN, Austin, TX (US); Yu ZHANG, Austin, TX (US)

(21) Appl. No.: 19/118,332

(22) PCT Filed: Oct. 4, 2023
(86) PCT No.: PCT/US2023/075967

§ 371 Date: Apr. 3, 2025

Related U.S. Application Data
(60) Provisional application No. 63/413,179, filed on Oct. 4, 2022.

(51) Int. Cl. B01L 3/00; B33Y 80/00; G01N 33/543; G01N 33/58

(52) U.S. Cl. CPC B01L 3/5085; B33Y 80/00; G01N 33/54386; G01N 33/582; B01L 2200/12; B01L 2300/0829; B01L 2300/12

(57) ABSTRACT

The present disclosure provides biological sensors that may be used to test for the presence or absence of an analyte. These biological sensors are prepared using additive manufacturing techniques to construct the base sensor shape and then functionalized with one or more types of recognition molecules. These biological sensors may be used to detect an abnormal state in a patient.

This application claims the benefit of priority to U.S. Provisional Application No. 63/413,179, filed on Oct. 4, 2022, the entire contents of which are hereby incorporated by reference.

### BACKGROUND

This invention was made with government support under Grant No. 7200AA20CA00019 awarded by the US Agency for International Development (USAID). The government has certain rights in the invention.

### 1. FIELD

The present disclosure relates generally to the field of diagnostics and diagnostic preparation. More particularly, it concerns compositions and biosensors made using a polymer matrix that maybe manufactured using additive manufacturing techniques such as stereolithography.

### 2. DESCRIPTION OF RELATED ART

3D printing, also referred to as additive manufacturing, is an emerging technology that holds great promise to revolutionize fabrication processes. It exemplifies the building of 3D objects through the digitally controlled deposition of the successive layers of materials such as metals, photopolymers, and thermoplastics.

A 3D printing procedure normally involves two key steps, designing the model and producing the model. The process begins with designing a virtual model of the object to be fabricated, through the use of a computer-aided design (CAD) drawing software or a 3D scanner. Then, the designed model is converted into stereolithographic (STL) format, which stores the parameters of the model as a series of coordinates of triangular sections, and transferred to the printer. Thereafter, in the 3D printer, the STL file is sliced to a certain number of 2D cross-sectional layers with the required layer thickness. In the end, the printer starts fabricating the 3D object by depositing the material layer-by-layer, based on the successful sequencing of the 2D cross-sectional layers.

Thus, the advantages of using the 3D printing method include reducing material depletion, reducing production costs, decreasing the lead time, ease and good replication due to the full automation fabricating method, the convenient flexibility of the design process, having various types of building materials, and constructing small and intricate objects with high precision.

The most common technique for linking ligands covalently to a hydrophilic solid surface is amine coupling through reactive esters. Many attempts in interaction analysis fail due to the non-functional or unsuccessful immobilization of a reactant onto the complex matrix of that surface. These problems are commonly related to either the molecular properties of ligand or analyte, the pH of the buffer, the type of surface, or the effectiveness of the regeneration after binding.

Therefore, there remains a need to develop new methods of preparing sensor systems that allow the advantages of additive manufacturing techniques to be utilized such as the constructions of unique shapes and allow for

### SUMMARY OF THE INVENTION

The present disclosure provides compositions for use in additive manufacturing for the preparation of biosensors. In some embodiments, the present disclosure provides a 3D printed biosensor with a lid and a testing probe. In some embodiments, the 3D printed biosensor described herein, wherein the testing probes have various shapes including cylinder shape, ball shape, “Janus” shape, or multi-parts assembling shape. In some embodiments, the 3D printed biosensor described herein, wherein the whole sensors are fabricated with a SLA resin which can be treated with polysaccharide for surface modification. In some embodiments, the 3D printed biosensor according to item 1, wherein the biosensor can test small molecular and large molecular at the same time. It is a multi-functional testing probe. In some embodiments, the 3D printed DNA biosensor described herein, wherein the designed bio-sensing approach was found to demonstrate superior selectivity against a non-complementary DNA target, with a detection range of 0.5-1000000 pmol per well (based on the surface area of the biosensor).

In another aspect, the present disclosure provides sensor systems comprising:
(A) a plurality of a shapes, wherein the shape comprises a length, width, and height, wherein the height is sufficient to allow the distribution of recognition molecules along the height of the shape;
(B) one or more recognition molecules along with the height of the shape such that the recognition molecules are homogenously dispersed along the height of the shape; and
(C) a surface, wherein the plurality of shapes is affixed onto a surface, wherein the surface is configured to fit to enclose one or more wells on a commercial well plate
   wherein the shape is formed using an additive manufacturing technique.

In some embodiments, the surface is configured to enclose all of the wells on the commercial well plate. In some embodiments, the commercial well plate is a 96 well plate. In other embodiments, the commercial well plate is 384 well plate. In other embodiments, the commercial well plate is a specially designed well plate. In some embodiments, the shapes are prepared using a biopolymer. In some embodiments, the biopolymer is further defined as:
(i) a dimethacrylate monomer;
(ii) a first PEGylated methacrylate monomer, wherein the PEG segment comprises an average molecular weight from about 600 to about 1,000; and
(iii) a photoinitiator.

In some embodiments, the biopolymer further comprises a second PEGylated methacrylate monomer, wherein the PEG segment comprises an average molecular weight from 100 to about 600. In some embodiments, the biopolymer is used to prepare the shape and the surface.

In some embodiments, the dimethacrylate monomer is joined by a linking group, wherein the linking group is an alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polymer of amino acid residues, a poly(ethylene glycol), a poly(propylene glycol), or a polycarbonate. In some embodiments, the linking group is alkanediyl_{(C≤12)}, substituted alkanediyl_{(C≤12)}, cycloalkanediyl_{(C≤12)}, substituted cycloalkanediyl_{(C≤12)}, heterocycloalkanediyl_{(C≤12)}, substituted heterocycloalkanediyl_{(C≤12)}, a poly(ethylene glycol), a poly(propylene glycol), or a polycarbonate. In some embodiments, the linking group is alkanediyl_{(C≤12)} or substituted alkanediyl_{(C≤12)}. In some embodiments, the linking group is alkanediylc_{(C≤12)}. In some embodiments, the linking group is 2,2,4-trimethylhexyl or 2,4,4-trimethylhexyl.

In some embodiments, the linking group further comprises two joining groups. In some embodiments, each of the joining groups is —O—, —NRₐ—, —C(O)—, —C(O)O—, —OC(O)—, —OC(O)O—, —C(O)NRₐ—, —NRₐC(O)—, —OC(O)NRₐ—, —NRₐC(O)O—, —S(O)ₐ—, —S(O)ₐO—, —OS(O)ₐ, —OS(O)ₐO—, wherein: Rₐ is hydrogen, alkyl_{(C≤6)}, or substituted alkyl_{(C≤6)}; and a is 0, 1, or 2. In some embodiments, each of the joining groups is —C(O)O—, —OC(O)—, —OC(O)O—, —C(O)NRₐ—, —NRₐC(O)—, —OC(O)NRₐ—, or —NRₐC(O)O—. In some embodiments, each of the joining groups is —OC(O)NRₐ— or —NRₐC(O)O—. In some embodiments, each of the joining group is —OC(O)NRₐ—. In some embodiments, each of the joining group is —OC(O)NH—. In some embodiments, the dimethacyrlate monomer is diurethane dimethacrylate. In some embodiments, the diurethane dimethacrylate is a mixture of multiple monomers.

In some embodiments, the first PEGylated methacrylate monomer comprises an average molecular weight of the PEG unit from about 600 to about 800 Daltons. In some embodiments, the first PEGylated methacrylate monomer, wherein the average molecular weight of the PEG unit is about 700 Daltons. In some embodiments, the second PEGylated methacrylate monomer comprises an average molecular weight of the PEG unit from about 400 to about 600 Daltons. In some embodiments, the second PEGylated methacrylate monomer comprises an average molecular weight of the PEG unit from about 500 to about 600 Daltons. In some embodiments, the second PEGylated methacrylate monomer, wherein the average molecular weight of the PEG unit is about 575 Daltons. In some embodiments, the photoinitiator is a phosphorus based photoinitiator. In some embodiments, the photoinitiator is a phosphorus oxide based photoinitiator. In some embodiments, the photoinitiator is diphenyl(2,4,6-trimethylbenzoyl)phosphine oxide (TPO).

In some embodiments, the biopolymer comprises from about 10 wt. % to about 65 wt. % of the dimethacrylate monomer in the pharmaceutical composition. In some embodiments, the biopolymer comprises from about 20 wt. % to about 40 wt. % of the dimethacrylate monomer in the biopolymer. In some embodiments, the biopolymer comprises from about 25 wt. % to about 35 wt. % of the dimethacrylate monomer in the pharmaceutical composition. In some embodiments, the biopolymer comprises from about 10 wt. % to about 55 wt. % of the first PEGylated methacrylate monomer in the biopolymer. In some embodiments, the biopolymer comprises from about 20 wt. % to about 40 wt. % of the first PEGylated methacrylate monomer in the biopolymer. In some embodiments, the biopolymer comprises from about 25 wt. % to about 35 wt. % of the first PEGylated methacrylate monomer in the biopolymer. In some embodiments, the biopolymer comprises from about 10 wt. % to about 55 wt. % of the second PEGylated methacrylate monomer in the biopolymer. In some embodiments, the biopolymer comprises from about 20 wt. % to about 40 wt. % of the second PEGylated methacrylate monomer in the biopolymer. In some embodiments, the biopolymer comprises from about 25 wt. % to about 35 wt. % of the second PEGylated methacrylate monomer in the biopolymer. In some embodiments, the biopolymer comprises from about 0.1 wt. % to about 5 wt. % of the photoinitiator in the biopolymer. In some embodiments, the biopolymer comprises from about 0.25 wt. % to about 3 wt. % of the photoinitiator in the biopolymer. In some embodiments, the biopolymer comprises from about 0.5 wt. % to about 2.5 wt. % of the photoinitiator in the biopolymer.

In some embodiments, the biopolymer comprises a ratio of the dimethacrylate monomer to the first PEGylated methacrylate monomer from about 5:1 to about 1:5. In some embodiments, the biopolymer comprises a ratio of the dimethacrylate monomer to the first PEGylated methacrylate monomer from about 2:1 to about 1:2. In some embodiments, the biopolymer comprises a ratio of the dimethacrylate monomer to the first PEGylated methacrylate monomer is about 2:1, 1:1, or 2:3. In some embodiments, the biopolymer comprises a ratio of the dimethacrylate monomer to the second PEGylated methacrylate monomer from about 5:1 to about 1:5. In some embodiments, the biopolymer comprises a ratio of the dimethacrylate monomer to the second PEGylated methacrylate monomer from about 2:1 to about 1:2. In some embodiments, the biopolymer comprises a ratio of the dimethacrylate monomer to the second PEGylated methacrylate monomer is about 2:1, 1:1, or 2:3. In some embodiments, the biopolymer comprises a ratio of the first PEGylated methacrylate monomer to the second PEGylated methacrylate monomer from about 5:1 to about 1:5. In some embodiments, the biopolymer comprises a ratio of the first PEGylated methacrylate monomer to the second PEGylated methacrylate monomer from about 2:1 to about 1:2. In some embodiments, the biopolymer comprises a ratio of the first PEGylated methacrylate monomer to the second PEGylated methacrylate monomer is about 1:1 or 1:2.

In some embodiments, the shape is a cylindrical shape. In some embodiments, the cylindrical shape comprises a height greater than its length or width. In some embodiments, the shape is a rectangular shape. In some embodiments, the rectangular shape comprises a height greater than its length or width. In some embodiments, the rectangular shape comprises an equal length and width. In some embodiments, the shape is a ball shape. In some embodiments, the ball shape comprises a spherical end. In some embodiments, the ball shape comprises a height longer than the width or length. In some embodiments, the ball shape comprises the spherical end on less than 50% of the height of the ball shape. In some embodiments, the shape comprises a first shape that is configured to bind to a second shape. In some embodiments, the first shape comprises a different recognition molecule than the second shape. In some embodiments, the shape is a custom shape for the application. In some embodiments, the shape is designed using CAD software.

In some embodiments, the shape further comprises a biologic polymer. In some embodiments, the biologic polymer is a peptide, nucleic acid, or a polysaccharide. In some embodiments, the biologic polymer is a polysaccharide. In some embodiments, the biologic polymer comprises one or more amine groups. In some embodiments, the biologic polymer is a polysaccharide, wherein the sugar group comprises one or more amine groups. In some embodiments, the biologic polymer is chitosan.

In some embodiments, the recognition molecule is covalently linked to the biologic polymer. In some embodiments, the covalent link between the biologic polymer and the recognition molecule is —Y₁-A₁-Y₂-A₂-Y₃—, wherein:
Y₁ is the biologic polymer;
Y₂ is alkanediyl_{(C≤12)}, alkenediyl_{(C≤12)}, arenediyl_{(C≤12)}, or a substituted version of any of these groups;
Y₃ is the recognition molecule; and
A₁ and A₂ are each independently selected from absent, —O—, —NRₐ—, —S—, —C(O)—, —CH(N—), —C(O)O—, —C(O)NH—, —OC(O)O—, —OC(O)NH—, —NHC(O)NH—, —C(NRₐ)O—, —C(NRₐ)NH—, —OC(NRₐ)O—, —OC(NRₐ)NH—, —NHC(NRₐ)NH—: wherein:
   Rₐ is hydrogen, alkyl_{(C≤6)}, or substituted alkyl_{(C≤6)}.

In some embodiments, Y₂ is alkanediyl_{(C≤12)} or substituted alkanediyl_{(C≤12)}. In some embodiments, Y₂ is alkanediyl_{(C≤12)}. In some embodiments, Y₂ is ethylene, propylene, or butylene. In some embodiments, Y₂ is propylene. In some embodiments, A₁ is —CH(N—). In some embodiments, A₂ is —CH(N—). In some embodiments, A₁ and A₂ are —CH(N—). In some embodiments, the recognition molecule is a biologic molecule. In some embodiments, the biologic molecule is an antibody, a sugar or polysaccharide, an amino acid or a polypeptide, or a nucleic acid or polynucleotide. In some embodiments, the biologic molecule is a polypeptide. In some embodiments, the polypeptide is a protein. In some embodiments, the protein is streptavidin. In other embodiments, the biologic molecule is a nucleic acid. In some embodiments, the nucleic acid is an aptamer. In other embodiments, the biological molecule is an antibody.

In some embodiments, the recognition molecule is a small molecule. In some embodiments, the recognition molecule further comprises a second recognition molecule. In some embodiments, the second recognition molecule is an antibody, a signaling molecule, a nucleic acid or polynucleotide, an amino acid or a polypeptide, a sugar or polysaccharide, or a small molecule. In some embodiments, the second recognition molecule is a small molecule. In some embodiments, the second recognition molecule is biotin. In some embodiments, the second recognition molecule is bound to a signaling molecule such as a dye. In some embodiments, the signaling molecule is a fluorophore.

In some embodiments, the recognition molecule binds to a macromolecule. In some embodiments, the macromolecule is a biologic molecule. In some embodiments, the biologic molecule is an antibody, a sugar or polysaccharide, an amino acid or a polypeptide, or a nucleic acid or polynucleotide. In some embodiments, the recognition molecule binds to a small molecule. In some embodiments, the small molecule is an active pharmaceutical ingredient, a co-factor, a vitamin, or a hormone. In some embodiments, the active pharmaceutical ingredient is a metabolite of the active pharmaceutical ingredient.

In some embodiments, the sensor system comprises a detection limit of less than 1 pmol per well. In some embodiments, the detection limit is less than 100 nmol per well. In some embodiments, the detection limit is less than 10 nmol per well. In some embodiments, the detection limit is less than 1 nmol per well. In some embodiments, the detection limit is less than 100 pmol per well. In some embodiments, the detection limit is less than 10 pmol per well. In some embodiments, the detection limit is less than 1 pmol per well. In some embodiments, the sensor systems have a detection limit from about 0.5 pmols to about 2 μmols per well for a 96 well plate. In some embodiments, the sensor systems have a detection limit from about 0.25 pmols to about 1 μmols per well for a 384 well plate].

In still another aspect, the present disclosure provides methods of detecting the presence of an analyte in a solution comprising reacting a solution containing the analyte to a sensor system described herein.

In some embodiments, the methods further comprise reacting the sensor system with a detection modality. In some embodiments, the detection modality is a dye labeled antibody such as a fluorophore. In some embodiments, the methods further comprise allowing the analyte to displace a detection modality from the sensor system; and washing the sensor system to remove the detection modality.

In some embodiments, the solution further comprises a buffer. In some embodiments, the buffer maintains the pH of the solution at from about 4 to about 10. In some embodiments, the pH of the solution is from about 6 to about 9. In some embodiments, the pH of the solution is from about 7 to about 8. In some embodiments, the pH of the solution is about 7.5. In some embodiments, the buffer is a sulfonic acid-based buffer. In some embodiments, the sulfonic acid-based buffer is HEPES or HEPBS. In some embodiments, the buffer is HEPBS. In some embodiments, the solution comprises a concentration of the buffer from about 0.1 mM to about 250 mM. In some embodiments, the concentration of the buffer is from about 1 mM to about 100 mM. In some embodiments, the concentration of the buffer is about 20 mM.

In some embodiments, the solution further comprises a salt. In some embodiments, the salt is a sodium salt. In some embodiments, the salt is sodium chloride. In some embodiments, the solution comprises a concentration of the salt from about 1 mM to about 25 M. In some embodiments, the concentration of the salt is from about 10 mM to about 10 M. In some embodiments, the concentration of the salt is about 1 M.

In some embodiments, the solution further comprises a surfactant. In some embodiments, the surfactant is a non-ionic surfactant. In some embodiments, the surfactant is Tween. In some embodiments, the surfactant is Tween 20. In some embodiments, the solution comprises an amount of the surfactant from about 0.0001% to about 1%. In some embodiments, the concentration of the surfactant is from about 0.001% to about 0.1%. In some embodiments, the concentration of the surfactant is about 0.01%.

In some embodiments, the solution further comprises a protein. In some embodiments, the protein is a serum albumin such as bovine serum albumin. In some embodiments, the solution further comprises a reducing agent. In some embodiments, the reducing agent is a borohydride based reducing agent such as sodium cyanoborohydride. In some embodiments, the solution comprises a concentration of the reducing agent from about 1 mM to about 1 M. In some embodiments, the concentration of reducing agent is from about 10 mM to about 500 mM. In some embodiments, the concentration of reducing agent is from about 50 mM to about 100 mM.

In still yet another aspect, the present disclosure provides methods of determining the presence of absence of a disease state in a patient comprising exposing a sample from the patient to the sensor system described herein.

In some embodiments, the methods determine the presence of a disease state. In other embodiments, the methods determine the absence of a disease state. In some embodiments, the patient is a mammal such as a human. In some embodiments, the sample is saliva, blood, urine, mucous, plasma, serum, sputum, stool, or tears.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the overall schematic images of ideal designed biosensor. Film shape, cylinder shape, ball shape, assembled “Janus” shape Thinker biosensor.

FIG. 2 shows the schematic images for fabricating process of the film biosensors.

FIGS. 3A & 3B show the curves analyses the testing sensitivity of biosensor. 3A large concentration range 40, 20, 10, 5, and 2.5 pmol/well. 3B concentration range from 2.5, 1.0, 0.5, 0.25, and 0.1 pmol/well.

FIG. 4 shows the schematic images for fabricating process of ball biosensors.

FIG. 5 shows the chemical reaction scheme for biosensor surface modification.

FIG. 6 shows the digital microscope images of Thinker from each reaction steps.

FIG. 7 shows the fluorescent microscope images of the ball bio-sensor, without biotin fluorescent, with biotin FITC, and DNA-FAM (from left to right).

FIG. 8 shows the chart/graph comparing different methods of the fluorescent intensity Vs concentration. The concentration was 20, 10, 5, 2.5, 1, 0.5, 0.25, and 0.1 pmol/well respectively. It shows that method 1 has the lowest reading, method 2 has the highest reading and the method 3 is the optimized reading.

FIG. 9 shows the buffer screening for control the non-specific bindings. Buffer A: 500 mM NaCl in DI-water; Buffer B: 2 M NaCl in DI-water; Buffer C: 20 mM HEPBS with 1 M NaCl and 0.01% tween 20, and Buffer D Tris 0.1 M NaCl. The pH of all buffers is around 7.5.

FIG. 10 shows the bar chart shows the binding percentage of Ball shape Thinker for 96 well plate. Buffer A-BSA/Buffer B-BSA with FAM-Biotin-DNA/FAM-DNA (1 pmol/per well).

FIG. 11 shows the schematic images show the assembling process of the “Janus” shaped Thinker biosensor.

FIG. 12 shows the morphology of the surface modified biosensors.

### DESCRIPTION OF ILLUSTRATIVE EMBODIMENTS

The present disclosure provides ways of adapting 3D printing technology to fabricate bio-sensors (Thinker) for biomedical applications. To this aim, film, cylinder shape, ball shape probes, “Janus” shape, and multi-parts assembly shape sensors were designed and 3D printed via the use of a laser stereolithographic method, using biocompatible polymeric resin described herein, in a layer-by-layer fashion based on the programmed computer aided design (CAD). Thereafter, the 3D printed probes are subjected to further modifications in order to construct the bio-sensing interface. Also, the biosensor can be assembled through different coupling techniques to detect both small and large molecules, and/or through streptavidin-biotin sandwich immobilization. The optimizing regeneration conditions are qualified as well.

The biosensor can be customized and designed with the help of computer-aided design (CAD) software. As is well known, the commercial immobilized surface well plate is a film-based 2D shape biosensor. There is a challenge that the UV plate reader may read inaccurate results due to the inconsistent fluorescent conversation in the well. It will miss leading the final diagnosis. However, the Thinker biosensors described herein are able to avoid this problem through their three-dimensional (3D) structure. The Thinker biosensors described herein consist of two parts: the first part being the lid on the top, and the other part is the functional probe.

With the help of CAD software, for the lid part, the Thinker biosensors fit in most of the commercial well-plates including 96 well-plates, 384 well-plates, and special shape well plates. For the functional probe, large surface area and multi-functional parts can be designed and adjusted. Herein, the lid includes design that avoids solvent evaporation step during the testing. Thus, 3D printed biosensor reduces the fabrication time and can produce custom made biosensor to satisfy various requirements.

The increasing amount of materials available for 3D printing is also an important advantage that enables the low-cost fabrication of objects made from functional materials. Composite materials containing a catalyst, electroactive or reactive component combined with the material with mechanical self-standing properties for incorporation into the 3D printed device is perhaps the most promising application of 3D printing in analytical chemistry. For example, instead of using the very expensive gold-coated surface, the Thinker biosensor uses an SLA resin called Bio-Flex and described herein. The Bio-Flex SLA resin contains a/multi liquid polymer (diluent components), a monomer, and a photo-polymerization initiator. In order to detect binding phenomena near the polymer layer of this surface, it is necessary to immobilize one of the molecules (the ligand) involved in the interaction onto the surface. Herein, low molecular weight chitosan was used for immobilization.

Furthermore, the present disclosure also provides methods of preparation of the sensor surface prior to immobilization and to help in choosing the correct coupling conditions. Immobilization procedures for ligands with primary amines or containing an aldehyde group are presented, as well as a method for immobilization of small molecules with a highly negative charge, which is more difficult. One example of a complex protocol is the multilayered streptavidin-biotin interaction often used in ELISA constructing from attaching streptavidin to the chitosan coating. Regeneration of the chip surface between measurements is a prerequisite for good quality data and long stability. A method is added for the optimization of regeneration conditions. The methods are given in a format adaptable to various types of SPR instruments and may need finetuning depending on the specifications of the equipment. These and more details will be described in more detail below.

### I. BIOSENSORS

In some aspects, the present disclosure provides biosensors that contain a biopolymer based shape that has been coated with a polysaccharide polymer such as chitosan to allow for the immobilization of one or more recognition molecules. In some embodiments, the biosensors is substantially, essentially, or entirely free of any other compound other than the biopolymer, a polysaccharide polymer, and one or more recognition molecules attached to the polysaccharide polymer.

The biosensors prepared herein may further comprise a biopolymer formulation with a particular shape. These shapes may include a ball, rod, cylinder, or other shape. In some embodiments, the shapes are a Janus shape, or a shape with two different recognition molecules on the shape. In some embodiments, the shapes have a length, width, or height. The height of the shape may be further

### A. Recognition Molecules

In some aspects, the present disclosure provides recognition molecules. The recognition molecules according to the embodiments may be, for example, an antibody, a growth factor, a hormone, a peptide, an aptamer, a small molecule such as a hormone, an imaging agent, or cofactor, or a cytokine. It has been demonstrated that the gp240 antigen is expressed in a variety of melanomas but not in normal tissues.

In certain additional embodiments, it is envisioned that cancer cell targeting moieties bind to multiple types of cancer cells. For example, the 8H9 monoclonal antibody and the single chain antibodies derived therefrom bind to a glycoprotein that is expressed on breast cancers, sarcomas and neuroblastomas (Onda, et al., 2004). Another example is the cell targeting agents described in U.S. Patent Publication No. 2004/005647 and in Winthrop, et al. (2003) that bind to MUC-1, an antigen that is expressed on a variety cancer types. Thus, it will be understood that in certain embodiments, cell targeting constructs according the embodiments may be targeted against a plurality of cancer or tumor types.

Additionally, certain cell surface molecules are highly expressed in tumor cells, including hormone receptors such as human chorionic gonadotropin receptor and gonadotropin releasing hormone receptor (Nechushtan et al., 1997). Therefore, the corresponding hormones may be used as the recognition molecule in the biosensors. Additionally, the recognition molecule that may be used include a cofactor, a sugar, a drug molecule, an imaging agent, or a fluorescent dye. Many cancerous cells are known to over express folate receptors and thus folic acid or other folate derivatives may be used as conjugates to trigger cell-specific interaction between the biosensors described herein and a cell (Campbell, et al., 1991; Weitman, et al., 1992).

Since a large number of cell surface receptors have been identified in hematopoietic cells of various lineages, ligands or antibodies specific for these receptors may be used as cell-specific targeting moieties. IL-2 may also be used as a cell-specific targeting moiety in a chimeric protein to target IL-2R+ cells. Alternatively, other molecules such as B7-1, B7-2 and CD40 may be used to specifically target activated T cells (The Leucocyte Antigen Facts Book, 1993, Barclay, et al. (eds.), Academic Press). Furthermore, B cells express CD19, CD40 and IL-4 receptor and may be targeted by moieties that bind these receptors, such as CD40 ligand, IL-4, IL-5, IL-6 and CD28. The elimination of immune cells such as T cells and B cells is particularly useful in the treatment of lymphoid tumors.

Other cytokines that may be used to target specific cell subsets include the interleukins (IL-1 through IL-15), granulocyte-colony stimulating factor, macrophage-colony stimulating factor, granulocyte-macrophage colony stimulating factor, leukemia inhibitory factor, tumor necrosis factor, transforming growth factor, epidermal growth factor, insulin-like growth factors, and/or fibroblast growth factor (Thompson (ed.), 1994, The Cytokine Handbook, Academic Press, San Diego). In some aspects, the targeting polypeptide is a cytokine that binds to the Fn14 receptor, such as TWEAK (see, e.g., Winkles, 2008; Zhou, et al., 2011 and Burkly, et al., 2007, incorporated herein by reference).

A skilled artisan recognizes that there are a variety of known cytokines, including hematopoietins (four-helix bundles) (such as EPO (erythropoietin), IL-2 (T-cell growth factor), IL-3 (multicolony CSF), IL-4 (BCGF-1, BSF-1), IL-5 (BCGF-2), IL-6 IL-4 (IFN-β2, BSF-2, BCDF), IL-7, IL-8, IL-9, IL-11, IL-13 (P600), G-CSF, IL-15 (T-cell growth factor), GM-CSF (granulocyte macrophage colony stimulating factor), OSM (OM, oncostatin M), and LIF (leukemia inhibitory factor)); interferons (such as IFN-γ, IFN-α, and IFN-β); immunoglobin superfamily (such as B7.1 (CD80), and B7.2 (B70, CD86)); TNF family (such as TNF-α (cachectin), TNF-0 (lymphotoxin, LT, LT-α), LT-0, CD40 ligand (CD40L), Fas ligand (FasL), CD27 ligand (CD27L), CD30 ligand (CD30L), and 4-1BBL)); and those unassigned to a particular family (such as TGF-β, IL 1α, IL-1β, IL-1 RA, IL-10 (cytokine synthesis inhibitor F), IL-12 (NK cell stimulatory factor), MIF, IL-16, IL-17 (mCTLA-8), and/or IL-18 (IGIF, interferon-γ inducing factor)). Furthermore, the Fc portion of the heavy chain of an antibody may be used to target Fc receptor-expressing cells such as the use of the Fe portion of an IgE antibody to target mast cells and basophils.

Furthermore, in some aspects, the recognition molecule may be a peptide sequence or a cyclic peptide. Examples, cell- and tissue-targeting peptides that may be used according to the embodiments are provided, for instance, in U.S. Pat. Nos. 6,232,287; 6,528,481; 7,452,964; 7,671,010; 7,781,565; 8,507,445; and 8,450,278, each of which is incorporated herein by reference.

Thus, in some embodiments, recognition molecules are antibodies or avimers. Antibodies and avimers can be generated against virtually any cell surface marker thus, providing a method for targeted to delivery of GrB to virtually any cell population of interest. These antibodies could also be used as fragments. Furthermore, the antibodies could have been developed in one type of animal and then humanized or developed using a human model. Methods for generating antibodies that may be used as cell targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the biosensors described herein may be conjugated to a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### B. Biopolymer Formulation

In some aspects, the present disclosure relates to compositions that may be used as resins to prepare devices and dosage forms. These compositions may contain a biopolymer formulation that contains a dimethacrylate monomer, one or more PEGylated diacrylate or dimethacrylate monomer, and a photoinitiator.

i. Dimethacrylate Monomer

In some aspects, the present disclosure comprises one or more dimethacrylate monomers that is linked by another functional group such as an alkyl chain. Without wishing to be bound by any theory, it is believed that these components may be used to impart hard structure to the composition. In particular, the dimethacrylate monomer may comprise a functional group between the two methacrylate units that is selected from: —O—, —NRₐ—, —C(O)—, —C(O)O—, —OC(O)—, —OC(O)O—, —C(O)NRₐ—, —NRₐC(O)—, —OC(O)NRₐ—, —NRₐC(O)O—, —S(O)ₐ—, —S(O)ₐO—, —OS(O)ₐ—, —OS(O)ₐO—, wherein: Rₐ is hydrogen, alkyl_{(C≤6)}, or substituted alkyl_{(C≤6)}; and a is 0, 1, or 2. These functional groups may join one or more alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polymer of amino acid residues, a poly(ethylene glycol), a poly(propylene glycol), or a polycarbonate. In particular, the methacrylate units can be joined by a pair of amides, carbamates, carbonates, or carboxylates. These units may be joined together by an alkanediyl or a substituted alkanediyl. A non-limiting example of a monomer is shown below:

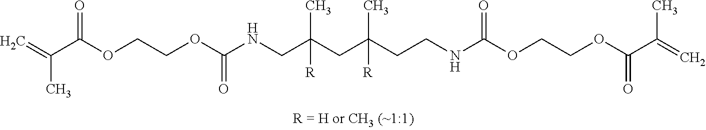

Furthermore, the present disclosure provides compositions that comprise from about 1.0% wt. to about 45% wt., from about 5.0% wt. to about 40% wt., from about 10.0% wt. to about 35% wt., or from about 20.0% wt. to about 30.0% wt. of each of the dimethacrylate monomers. In some aspects, the amount of the dimethacrylate monomers may be from about 1% wt., 2.5% wt., 5% wt., 7.5% wt., 10% wt., 12.5% wt., 15% wt., 17.5% wt., 20% wt., 22.5% wt., 25% wt., 27.5% wt., 30% wt., 32.5% wt., 35% wt., 37.5% wt., 40% wt., 42.5% wt., 45% wt., to about 50% wt., or any range derivable therein.

ii. PEGylated Diacrylate Monomer

As used herein, the present composition may comprise one or more PEGylated diacrylate monomer. In some embodiments, the composition may comprise a single PEGylated diacrylate or dimethacylrate monomer. In other embodiments, the composition comprises two or more PEGylated diacrylate or dimethacrylate monomers. If the composition comprises two or more PEGylated diacrylate or dimethacrylate monomers, then each PEGylated diacrylate or dimethacrylate monomers are different such as having different PEG lengths.

Without wishing to be bound by any theory, it is believed that the PEG segments should have a length of less than 1000 Daltons and more than 100 Daltons. Outside of these sizes, the PEGylated monomer may show the appropriate properties to create a device that shows sufficient flexibility to be used. In particular, the PEGylated diacrylate or dimethacrylate monomers may each have a PEG segment from about 100, 200, 300, 400, 500, 600, 700, 800, 900, or 1,000 Daltons. In particular, if two or more PEG segments are used, the composition comprises a PEGylated diacrylate or dimethacrylate monomers with a PEG segment of greater than 600 Daltons and a second PEGylated diacrylate or dimethacrylate monomers with a PEG segment of less than 600 Daltons. In particular, the PEG segment in the first PEGylated diacrylate or dimethacrylate monomers may be from about 600, 650, 700, 750, 800, 850, 900, 950, or 1,000 Daltons, or any range derivable therein. In other embodiments, the second PEGylated diacrylate or dimethacrylate monomers may have a PEG segment with a weight from about 100, 150, 200, 250, 300, 350, 375, 400, 425, 450, 475, 500, 525, 550, 575, or 600 Daltons, or any range derivable therein.

Furthermore, the present disclosure provides compositions that comprise from about 1.0% wt. to about 45% wt., from about 5.0% wt. to about 40% wt., from about 10.0% wt. to about 35% wt., or from about 20.0% wt. to about 30.0% wt. of each of the PEGylated diacrylate or dimethacrylate monomers. In some aspects, the amount of the PEGylated diacrylate or dimethacrylate monomers may be from about 1% wt., 2.5% wt., 5% wt., 7.5% wt., 10% wt., 12.5% wt., 15% wt., 17.5% wt., 20% wt., 22.5% wt., 25% wt., 27.5% wt., 30% wt., 32.5% wt., 35% wt., 37.5% wt., 40% wt., 42.5% wt., 45% wt., to about 50% wt., or any range derivable therein.

iii. Photoinitiators

The photoinitiators described herein refer to a substance that after being illuminated by light, is configured to initiate or enhance a chemical reaction (although the substance itself may not undergo a reaction). In some embodiments, the photoinitiator catalyzes the reaction/cross-linking of the SLA resin components, when activated by an energy source (e.g. light or laser beam) during an AM process. In one embodiment, the photoinitiator is an antimony-free photoinitiator. In some embodiments, the antimony-free photoinitiator is configured to reduce, prevent, and/or eliminate residue after burning out of the SLA resin. In some embodiments, the photoinitiator is a diphenyl (2,4,6-trimethyl benzoyl) phosphine oxide (TPO), commercially available TPO 415952 (Sigma-Aldrich). In some embodiments, the present disclosure provides compositions with from about 0.01 wt. % to about 10 wt. %, from about 0.025 wt. % to about 5 wt. %, or from about 0.05 wt. % to about 2 wt. % of a photoinitiator component in SLA resin. The amount of the photoinitiator is from about 0.01 wt. %, 0.025 wt. %, 0.05 wt. %, 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1.0 wt. %, 1.25 wt. %, 1.5 wt. %, 1.75 wt. %, 2.0 wt. %, 2.5 wt. %, 3.0 wt. %, 3.5 wt. %, 4.0 wt. %, 4.5 wt. %, 5.0 wt. %, 6.0 wt. %, 7.0 wt. %, 8.0 wt. %, 9.0 wt. %, to about 10.0 wt. %, or any range derivable therein. The photoinitiator may be pre-dissolved in amount of solvent that is equal to the weight of the composition or more. The solvent used may be an alcoholic solvent that comprises from 1 to 6 carbon atoms such as methanol, ethanol, isopropanol, propanol, butanol, or tert-butanol.

In other embodiments, the TPO is a water-soluble, commercially available water-soluble TPO-based nanoparticle photoinitiator, contains ionic surfactant 906808 (Sigma-Aldrich), and water-soluble TPO based nanoparticle photoinitiator, contains nonionic surfactant 906816 (Sigma-Aldrich).

iv. Other Excipients

In some aspects, the present disclosure provides biopolymer that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the biosensor. It is also possible to have more than one active agent in a given solution so that the particles formed contain more than one recognition molecule. In particular, the compositions may further comprise one or more flowability excipients such as a silicon compound. The silicon compound may include an oxide of silicon such as silicon dioxide.

Any pharmaceutically acceptable excipient known to those of skill in the art may be used to produce the biosensors disclosed herein. Examples of excipients for use with the present disclosure include, lignin, gelatin methacrylate, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerin and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the biosensors to enhance the efficacy and efficiency of the recognition molecule in the biosensor. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include: sucrose, trehalose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidines, and tyloxapol. In particular, the composition may further comprise one or more silicon compounds such as silicon dioxide that improves the flowability of the composition.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing, complexing agent, fragrance, preservative or a combination thereof.

Compositions with enhanced solubility may comprise a mixture of the recognition molecule and an additive that enhances the solubility of the recognition molecule. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the biosensors may further comprise one or more surfactants. Surfactants that can be used in the disclosed biosensors to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a biosensor of the present disclosure compared to peak solubility of the reference standard agent under the same conditions. Preferably, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

In other aspects, the present biosensors may further comprise one or more opacifying agents. Opacifying agents include such compounds as titanium oxide and alter the clarity and ability of energy to be absorbed by the biosensors. Alternatively, these biosensors may alter the amount of energy needed to achieve appropriate processing of the compositions. Some non-limiting examples of opacifying agents include those taught by U.S. Pat. Nos. 4,009,139, 5,571,334, and PCT Patent Application No. WO 2020/122950, the entire contents of which are hereby incorporated by reference. Some non-limiting examples of opacifying agents including Aerosil®, Cab-O Si®, or other silicon dioxides, aluminum hydroxide, alumina, aluminum silicate, arachidic acid, barium sulfate, bentonite, calamine, calcium carbonate, calcium phosphate dibasic, calcium phosphate tribasic, calcium silicate, calcium sulfate, ceric oxide, acetyl alcohol, activated charcoal, charcoal, diatomaceous earth, erucamide, ethylene glycol monosterate, Fuller's earth, guanine, hectorite, kaolin, magnesium aluminum silicate, magnesium carbonate, magnesium oxide, magnesium phosphate tribasic, magnesium silicate, magnesium trisilicate, myristic acid, palmitic acid, silica, stannic oxide, stearic acid amide, stearoyl monoethanolamine sterate, stearyl palmitate, talc, titanium dioxide, Veegum® or other granular magnesium aluminum silicates, zinc carbonate basic, zirconium oxide, or zirconium silicate. In other aspects, these excipients may be a light absorbing excipient such as a dye or fluorophore. Some non-limiting examples of dyes include xanthene, BOPIDY, coumarin, cypate, or other well-known conjugated systems of multiple bonds.

In some aspects, the amount of the excipient in the biosensor is from about 0.1% to about 20% w/w, from about 0.25% to about 10% w/w, from about 0.5% to about 7.5% w/w, or from about 0.5% to about 5% w/w. The amount of the excipient in the pharmaceutical composition comprises from about 0.1%, 0.2%, 0.25%, 0.3%, 0.4%, 0.5%, 0.6%, 0.7%, 0.75%, 0.8%, 0.9%, 1%, 1.25%, 1.5%, 1.5%, 1.75%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total biosensor weight. In one embodiment, the amount of the excipient in the biosensor is at 0.25% to 2.5% w/w of the total weight of the biosensor.

### C. Polysaccharide Polymer

In some aspects, the biosensors described herein may be coated with a polysaccharide polymer such as chitosan. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit) (Varum et al., 1991). The chitosan has a positive charge, stemming from the de-acetylated amino group (—NH₃⁺). Chitosan, chitosan derivatives, or salts (e.g., nitrate, phosphate, sulphate, hydrochloride, glutamate, lactate or acetate salts) of chitosan may be used and are included within the meaning of the term “chitosan.” As used herein, the term “chitosan derivatives” is intended to include ester, ether, or other derivatives formed by bonding of acyl and/or alkyl groups with —OH groups, but not the NH₂ groups, of chitosan. Examples are O-alkyl ethers of chitosan and O-acyl esters of chitosan. Modified chitosans, particularly those conjugated to polyethylene glycol, are also considered “chitosan derivatives.” Many chitosans and their salts and derivatives are commercially available (e.g., SigmaAldrich, Milwaukee, WI). In preferred aspects, chitosan nanoparticles of the embodiments are PEGylated.

Methods of preparing chitosan and their derivatives and salts are also known, such as boiling chitin in concentrated alkali (50% w/v) for several hours. This produces chitosan wherein 70%-75% of the N-acetyl groups have been removed. A non-limiting example of a chitosan, wherein all of the N-acetyl groups have been removed, is shown below:

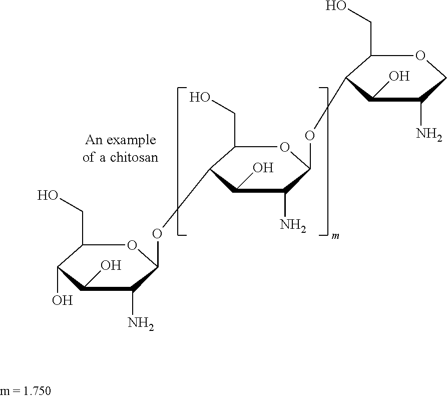

Chitosans may be obtained from any source known to those of ordinary skill in the art. For example, chitosans may be obtained from commercial sources. Chitosans may be obtained from chitin, the second most abundant biopolymer in nature. Chitosan is prepared by N-deacetylation of chitin. Chitosan is commercially available in a wide variety of molecular weight (e.g., 10-1000 kDa) and usually has a degree of deacetylation ranging between 70%-90%.

The chitosan used herein may have a molecular weight from about 1000 Daltons to about 100,000 Daltons. The weight may be from about 1000, 2000, 3000, 4000, 5000, 6000, 7000, 8000, 9000, 10,000, 20,000, 25,000, 30,000, 40,000, 50,000, 60,000, 70,000, 75,000, 80,000, 90,000, to about 100,000 Daltons, or any range derivable therein. Chitosans of different molecular weights can be prepared by enzymatic degradation of high molecular weight chitosan using chitosanase or by the addition of nitrous acid. Both procedures are well known to those skilled in the art and are described in various publications (Li et al., 1995; Allan and Peyron, 1995; Domard and Cartier, 1989). The chitosan is water-soluble and may be produced from chitin by deacetylation to a degree of greater than 40%, preferably between 50% and 98%, and more preferably between 70% and 90%.

Some methods of producing chitosan involve recovery from microbial biomass, such as the methods taught by U.S. Pat. No. 4,806,474 and U.S. Patent Application No. 2005/0042735, herein incorporated by reference. Another method, taught by U.S. Pat. No. 4,282,351, teaches only how to create a chitosan-beta-glucan complex.

The chitosan, chitosan derivative, or salt used herein is water soluble. Chitosan glutamate is water soluble. By “water soluble” it is meant that that the chitosan, chitosan derivative, or salt dissolves in water at an amount of at least 10 mg/ml at room temperature and atmospheric pressure. The chitosan, chitosan derivative, or salt used in the present invention has a positive charge.

Additional information regarding chitosan and chitosan derivatives can be found in U.S. Patent App. Pub. Nos. 2007/0167400, 2007/0116767, 2007/0311468, 2006/0277632, 2006/0189573, 2006/0094666, 2005/0245482, 2005/0226938, 2004/0247632, and 2003/0129730, each of which is herein specifically incorporated by reference.

### II. ADDITIVE MANUFACTURING METHODS

Various additive manufacturing including additive systems are contemplated in the present disclosure. These additive manufacturing or additive systems represent machines used for additive manufacturing. These machines operate under various process categories and are used to refer to a category of machines rather than a particular commercial vendor variation of process methodology. Several examples of additive manufacturing process categories include: binder jetting, directed energy deposition, material extrusion, material jetting, powder bed fusion, sheet lamination, and vat photopolymerization. Vat photopolymerization refers to an additive manufacturing process in which liquid photopolymer in a vat is selectively cured by light-activated polymerization. Similarly, stereolithography is a vat photopolymerization process used to produce parts from photopolymer materials in a liquid state using one or more lasers to selectively cure to a predetermined thickness and harden the material into shape layer upon layers. In particular, stereolithography apparatus is an SLA additive machine. As a non-limiting example, SL additive machines are commercially available via various vendors, such as Envision Tec 3D, Formlabs, 3D systems corporation, etc.

The devices described herein are cured products of the resin composition for stereolithography. Specifically, the flexible AM devices of the present invention are cured by irradiating the polymer composition for stereolithography with laser/light. While the hardness, tensile strength at break, tensile elongation at break, and compression of the flexible shaped biomedical devices made using the pharmaceutical composition may be set appropriately according to the mechanical properties required for the product.

The flexible shaped biomedical devices made according to the methods described herein may be formed into any desired shapes by a stereolithography method. These flexible shaped biomedical devices may be produced by any known stereolithography methods, using the compositions described herein as raw material.

The method for producing the flexible biomedical devices can be suitably performed by using the composition described herein, in a conventionally known stereolithography method that uses a liquid resin as a raw material, such as being used in various stereolithography methods including LCD (stereolithography liquid display method: Liquid Crystal Display), DLP (stereolithography projector (surface exposure) method: Digital Light Processing), and SLA (stereolithography laser method: Stereolithography Apparatus).

In some aspects, the present disclosure provides composition that may be used to prepare biomedical devices described herein. These devices are prepared using a method including the steps of forming a first-layer cured product by supplying the compositions for stereolithography described herein onto a stereolithography platform, and irradiating the polymer composition for stereolithography with light/laser to cure the polymer composition for stereolithography; forming a second-layer cured product by supplying the composition described gereub for producing the second-layer cured product onto the first-layer cured product, and irradiating the compositions for stereolithography with laser/light to cure the polymer composition for stereolithography; and repeating the same step as the step of forming the second-layer cured product until a final layer is formed to devices (AM parts) with a three-dimensional shape. For a stereolithography method, any known 3D printer may be used and several types of 3D stereolithographic printer are commercially available.

In a stereolithography method, the thickness of a single layer upon curing the composition described herein is, for example, from about 1 micron to about 1 mm, from about 10 to about 750 microns, from about 20 to about 500 microns, or from about 25 to about 300 microns. The size of the layers may be from about 1 m, 2.5 μm, 5 μm, 10 m, 15 m, 20 μm, 25 μm, 50 μm, 75 μm, 100 μm, 150 μm, 200 μm, 250 μm, 300 μm, 350 μm, 400 μm, 450 μm, 500 μm, 600 μm, 700 μm, 800 μm, 900 μm, to about 1 mm, or any range derivable therein The irradiation light is typically ultraviolet light and preferably includes light with a wavelength of 405 nm. The wavelength of light may be from about 300 nm to about 800 nm, from about 350 nm to about 600 nm, or from about 400 nm to about 500 nm. Similarly, the laser used may have a power from about 10 mW to about 1 W, from about 50 nW to about 750 mW, or from about 100 mW to about 500 mW. The irradiation laser power is 250 mW. The irradiation time for curing a single layer of the composition depends on the stereolithography method and may be adjusted appropriately. For example, in the DLP method, the irradiation time is about 1 to 60 seconds, but again depends on the specific formulation and method of stereolithography. These devices may be produced in an environment at about room temperature (e.g., 15 to 35 °C).

After the stereolithography, optionally, a general secondary treatment, such as high-pressure mercury lamp irradiation, metal halide lamp irradiation, UV-LED irradiation, or heating, may be additionally performed. The secondary treatment can modify the surface after stereolithography, improve the strength, or accelerate curing. The secondary treatment can be performed in combination with stereolithography, although the secondary treatment is not necessarily required, depending on the stereolithography conditions.

In particular, when UV-LED irradiation is employed as a secondary treatment in the method of preparing a device described herein, any unreacted monomer contained in the AM parts can be cured by radicals generated from the photopolymerization initiator, which can further increase the mechanical strength.

In other aspects, the biosensors described herein may also be used in an additive manufacturing platform. Some of the additive manufacturing platforms that may be used herein include 3D printing such as stereolithography may be used to obtain the final biosensor.

In some aspects, the biosensors described herein are processed in a final dosage form instead of as a device. The granules that are produced by the process may be further processed into a capsule or a tablet. Before formulation into a capsule or tablet, the granule may be further milled before being compressed into the capsule or tablet.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active pharmaceutical ingredient”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “sensor,” “biosensor,” “compositions,” “pharmaceutical compositions,” “formulations,” “pharmaceutical formulations,” “preparations”, and “pharmaceutical preparations” are used synonymously and interchangeably herein.

As used herein, “SLA resin” refers to a stereolithographic resin. For example, SLA resins can be utilized as a building material and/or feedstock used in conjunction with vat polymerization and SLA. The conventional resin which can be applied to SLA equipment mainly focus on producing suitably elastic shaped part or devices. The mechanism properties were their top concerning properties to fit their industrial applications such as those described in WO 2017/154335.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aliphatic, aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “degradation” or “chemically sensitive” refers to a compound that is destroyed or rendered inactive and unacceptable for use. Degradation may include compounds which have one or more chemical bonds present in the compound has been broken.

The term “dissolution” as used herein refers to a process by which a solid substance, such as the active ingredients or one or more excipients, is dispersed in molecular form in a medium. The dissolution rate of the active ingredients of the pharmaceutical dose of the invention is defined by the amount of drug substance that goes in solution per unit time under standardized conditions of liquid/solid interface, temperature and solvent composition.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

A “poorly soluble drug” refers to a drug which meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified components has been purposefully formulated into a composition and/or is present only as a contaminant or in trace amounts. The total amount of all containments, by-products, and other material is present in that composition in an amount of less than 2%. The term “essentially free of” or “essentially free” is used to represent that the composition contains less than 1% of the specific component. The term “entirely free of” or “entirely free” contains less than 0.1% of the specific component.

As used herein, the term “substantially intact” in terms of a specified component, is used herein to mean that the specified component has not been degraded or rendered inactive in an amount less than 5%. The term “essentially intact” is used to represent that less than 2% of the specific component has been degraded or rendered inactive. The term “entirely intact” contains less than 0.1% of the specific component that has been degraded or rendered inactive.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the electromagnetic energy absorbing excipients are adjacent to another atom of the electromagnetic energy absorbing excipients.

The terms “substantially” or “approximately” as used herein may be applied to modify any quantitative comparison, value, measurement, or other representation that could permissibly vary without resulting in a change in the basic function to which it is related.

A temperature, when used without any other modifier, refers to room temperature, preferably 23 °C unless otherwise noted. An elevated temperature is a temperature which is more than 5 °C greater than room temperature; preferably more than 10 °C greater than room temperature.

The term “unit dose” refers to a formulation of the pharmaceutical composition such that the formulation is prepared in a manner sufficient to provide a single therapeutically effective dose of the active agent to a patient in a single administration. Such unit dose formulations that may be used include but are not limited to a single tablet, capsule, or other oral formulations, or a single vial with a syringeable liquid or other injectable formulations. In some forms, the final pharmaceutical composition that is produced is no longer a powder and is further produced as a homogenous final product.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Medical Devices Via Basic Biomedical SLA Resin

During an additive manufacturing process, the energy source (e.g. laser) polymerizes the SLA resin in successive layers in an additive manufacturing build to form an additive manufacturing preform. When a layer is completed, a leveling blade is moved across the surface in order to smooth the surface before the next layer of additive manufacturing feedstock material is deposited. After the blade smooths the surface, the platform is lowered by a distance equal to the layer thickness of a build layer. Then the energy source again tracks the build pattern and cures the SLA resin in designated areas, adding another build layer to the additive manufacturing build. This process of tracing is repeated until the additive manufacturing build is complete, forming the additive manufacturing preform. After the additive manufacturing build is completed, then the additive manufacturing perform undergoes a post-cure step to finish the additive manufacturing part.

The preferable viscosity of the resin ink for SLA is around 700 mPa·S or less, as measured using a rotational viscometer-Visco-Tester in an air environment at a temperature of 25 °C and relative humidity of 35%. The viscosity of the purchased commercial DUDMA, PEGDA575, and PEGDA700 is ~2300 mPa·S, ~75 mPa·S, and ~95 mPa·S, respectively.

The viscosity of the mixed resin (item 1) is approximately 600 mPa·S. The lower the processing temperature during printing, the higher the viscosity of the final resin ink.

The thermal properties of the raw materials and physical mixtures were determined through a Mettler-Toledo TGA/DSC1 analyzer (Mettler-Toledo, Schwerzenbach, Switzerland). Pure TPO, DUDMA, PEGDA 575, and PEGDA 700, and all samples were ramped from 35 to 350 °C at a rate of 20 °C/min. The furnace was purged using ultra-purified nitrogen at a flow rate of 50 mL/min. The STAR software was used to operate the instrument and collect the data, while data were analyzed using Microsoft Excel software (Version 2007, Microsoft, Redmond, WA, USA). From the TGA data, it's evident that all of our excipients and thus formulations are stable in the processing temperature used.

Differential scanning calorimetry (DSC, DSC Q20, TA® instruments, New Castle, DE, USA) analysis was used to characterize the pure active functional ingredient (AFI), polymer, and AFI-polymer physical mixture. Approximately 7-12 mg of samples were weighed in standard DSC aluminum pans and sealed with standard aluminum lids (DSC consumables incorporated, Austin, MN, USA) using a calibrated balance. The prepared samples were subjected to a heat-cool-heat ramp circle heated from 25 °C to 200 °C with a ramp rate of 5 °C/min. A purge gas (Nitrogen) at a flow rate of 50 mL/min was used for all the experiments. The data were collected by TA advantage software (Q series, Version 2007 build 13029.20308) and analyzed by TA instruments Universal Analysis 2000. The results were presented as a plot of temperature (°C) versus reverse heat flow (mW).

The DSC thermograms of bulk excipients demonstrated that TPO exhibited an endothermic thermal transition at 98 °C due to its melting. Our developed formulation didn't exhibit any endotherm at around the range of processing temperature owing to the liquid nature of the polymeric substances used in the formulations.

In order to additively build apart, the energy source traces out successive cross-sections of a three-dimensional object in a vat of liquid photosensitive polymer. The resin crosslinks to form a thermoset polymer, while the excess resin remains liquid resin adjacent to the additive manufacturing build. Once the additive manufacturing build is completed, the additive manufacturing build is elevated and drained to remove excess SLA resin. Once the draining is completed, the final cure is completed by placing the part (or parts) into a UV oven or conveyor and subjecting the additive manufacturing parts to a sufficient amount of light for a sufficient amount of time to cure the thermoset polymer.

An SLA resin composition is prepared according to the above procedure. Optionally, the SLA resin is degassed by degassing sonication in a temperature-controlled water tank. The SLA resin is configured into an additive machine configured to utilize SLA resin as the AM build material.

Examples of liquid resin compositions: liquid resin compositions were prepared by feeding the components shown in Table 1 to a container and stirring the mixture at room temperature for 5 mins.

Test specimens of examples 1 to 3 were prepared from Table 1 listed resin compositions for evaluation according to the following method. Laser beams were selectively applied to the resin composition at a laser power at the irradiation surface of a 250 mW laser and a scanning speed at which the cure depth of each composition was 100 microns using a Form2 3D printer (manufactured by Formlabs, USA) to form a cured resin layer. This step was repeated to obtain a test specimen.

TABLE 1
Basic SLA Resin Formulation

| Ingredients (wt. %) | Example 1 | Example 2 | Example 3 | |
|---|---|---|---|---|
| DUDMA | 44.5 (1) | 39.4 (2) | 24.5 | (2) |
| PEGDA 575 | 44.5 (1) | 39.4 (2) | 36.75 | (3) |
| PEGDA 700 | | 19.7 (1) | 36.75 | (3) |

| | | | |
|---|---|---|---|
| TPO | 1 | 1.5 | 2 |

### Example 2-Preparation of Biosensors

The Biosensor was prepared using FDA tested biomedical polymer di-urethane di-methacrylate (DUDMA), Polyethylene glycol diacrylate (PEGDA 575, PEGDA 700), and a photoinitiator (Diphenyl (2,4,6-trimethyl benzoyl) phosphine oxide (TPO) are selected for producing the structure of the Thinker biosensor. These compositions are used as described above.

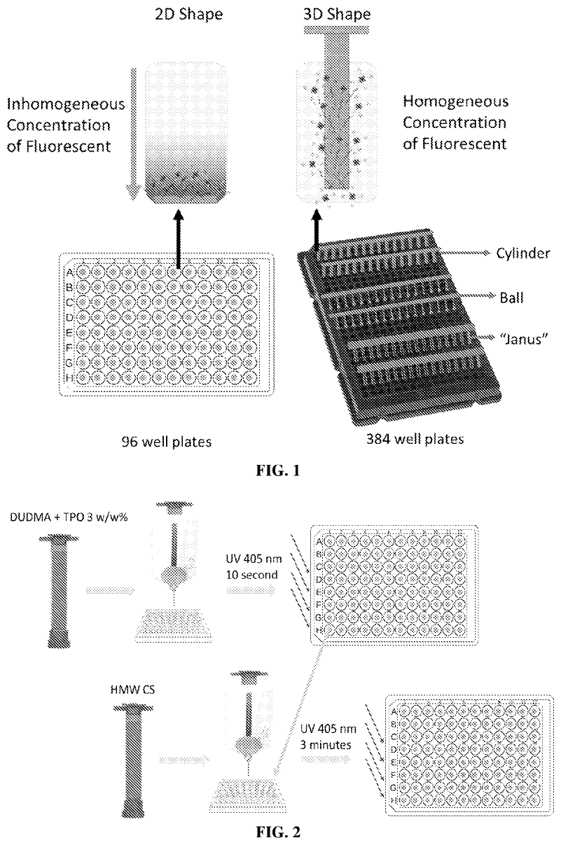

The overall schematic images of ideal designed biosensor are shown in FIG. 1 including film shape, cylinder shape, ball shape, assembled “Janus” shape Thinker biosensor. For the film biosensor, a bioprinter bioX was used to fabricate the biosensor. For the other complex 3D shaped biosensors, a stereolithography printer (Form2, FormLab int. USA) was used for the manufacture.

FIG. 2 illustrates a schematic for fabricating process of the film biosensors. First of all, to facilitate the subsequent covalent immobilization of DNA recognition element onto the 3D printed probe, via glutaraldehyde reaction with amine. The probes were first immerged into chitosan (CH) solution and then UV curved for 3 min at 60 °C.

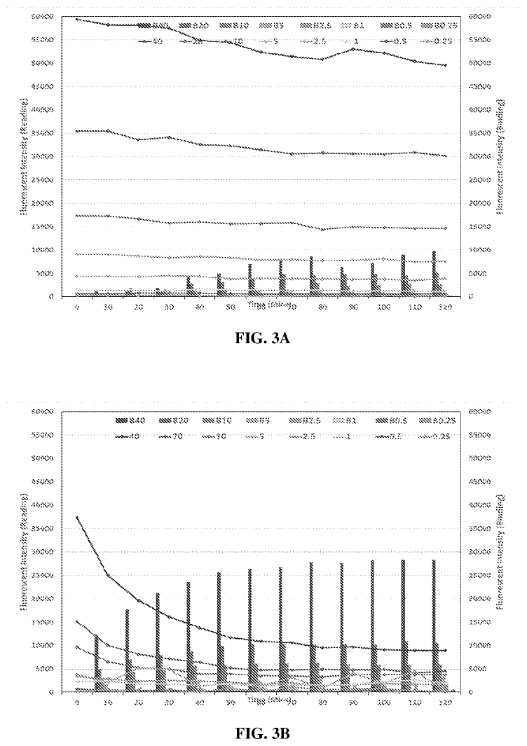

The curves analyses to test the sensitivity of biosensor is shown in FIG. 3. FIG. 3A demonstrate the detection of the biosensor at large concentration range 40, 20, 10, 5, and 2.5 pmol/well. Then, FIG. 3B shows the detection of the biosensor at low concentration range from 2.5, 1.0, 0.5, 0.25, and 0.1 pmol/well. The results show that the biosensor shows good detection sensitivity start from 0.5 pmol/well of the biosensor at a surface area of biosensor at 47.

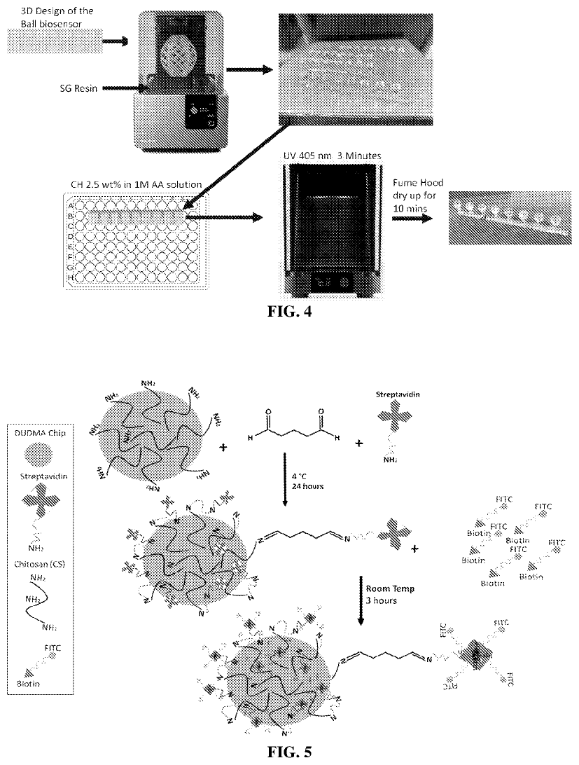

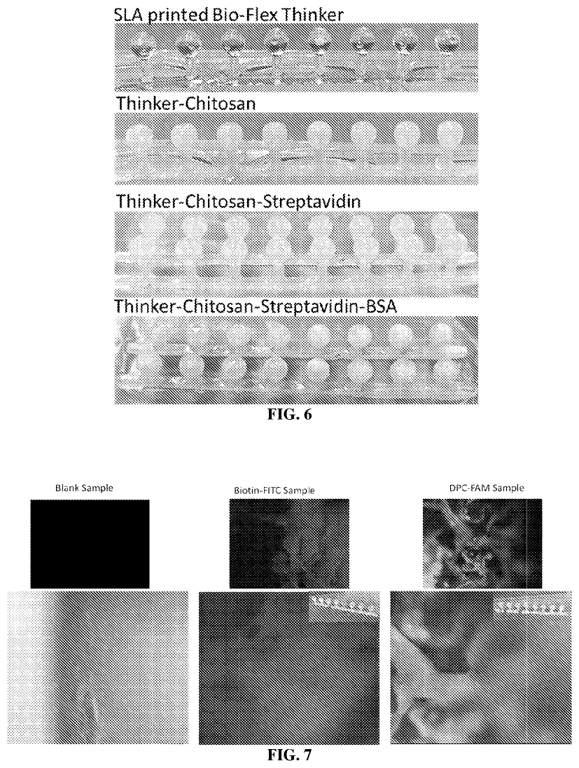

The images for fabricating process of ball biosensors are shown in FIG. 4. Then, the chemical reaction scheme for biosensor surface modification is shown in FIG. 5. The CH films were cross-linked by immersion in 0.5% (w/v) GA solution from Tris buffer at pH 7.5. The crosslinking procedure was allowed to continue for 24 h at 4 °C. Subsequently, these products were thoroughly washed with deionized water and finally dehydrated again by slow drying under a laminar flow hood. Some CH films without any crosslinking were taken as reference materials. FIG. 6 shows the microscope images of Thinker from each reaction steps.

The fluorescent microscope images of the ball biosensors are shown in FIG. 7. From left to the right in FIG. 7 show the ball biosensor without biotin fluorescent, with biotin FITC, and DNA-FAM, separately.

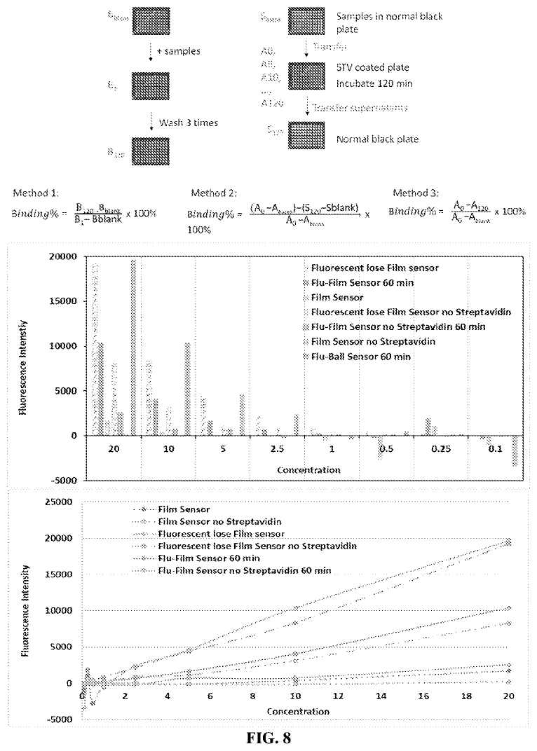

Analysis of different methods are described and plotted as the fluorescent intensity vs concentration. The concentration was 20, 10, 5, 2.5, 1, 0.5, 0.25, and 0.1 pmol/well respectively. This data is shown in FIG. 8. It shows that method 1 has the lowest reading, method 2 has the highest reading and the method 3 is the optimized reading.

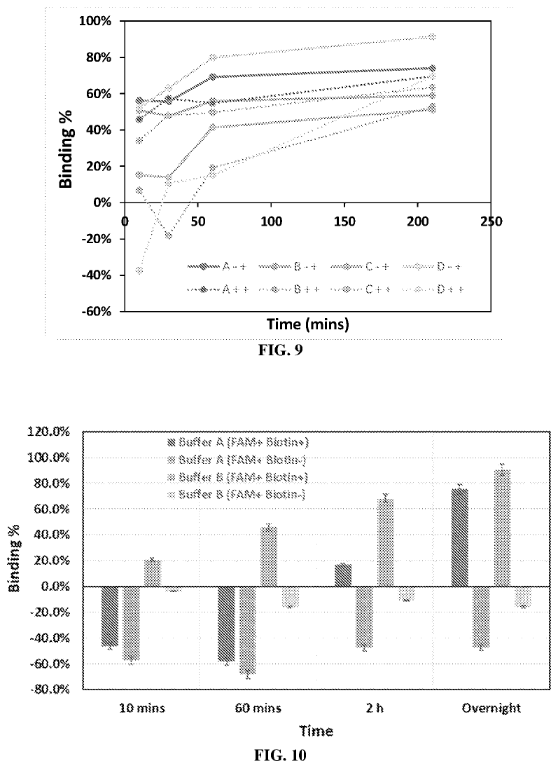

Similarly, the use of different buffers was screened to control for different non-specific binding. The results of the screen are shown in FIG. 9. Buffer A: 500 mM NaCl in DI-water; Buffer B: 2 M NaCl in DI-water; Buffer C: 20 mM HEPBS with 1 M NaCl and 0.01% tween 20, and Buffer D Tris 0.1 M NaCl. pH of all buffers is around 7.5. Basic Buffer for washing: 20 mM HEPBS. Herein, the buffer C shows the optimized control for controlling the non-specific bindings. FIG. 10 shows the bar chart shows the binding percentage of Ball shape Thinker for 96 well plate. Buffer A-BSA/Buffer B-BSA with FAM-Biotin-DNA/FAM-DNA (1 pmol/per well). The binding percentage of the Thinker with the right parameters was able to reach 100%.

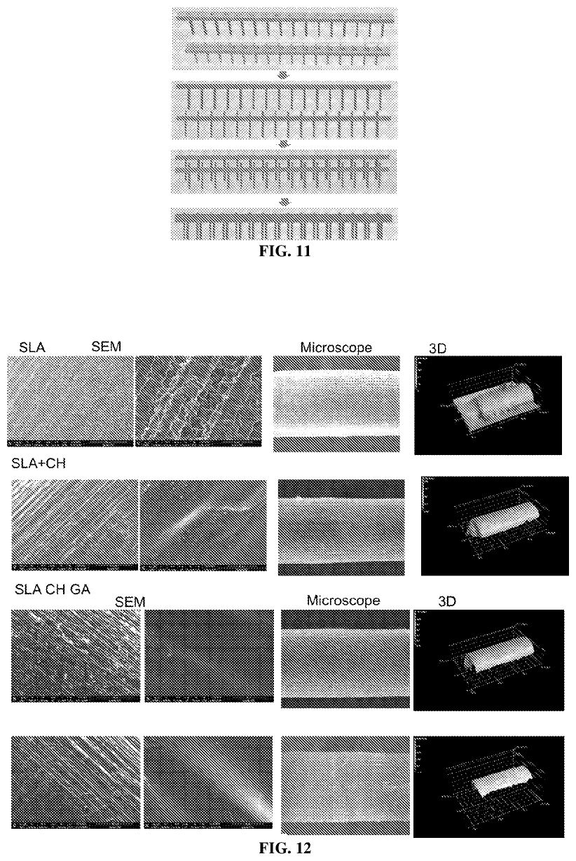

Finally, the schematic process of assembling the “Janus” shaped Thinker biosensor is shown in FIG. 11. To analyze both large molecules and small molecules at the same time, the two parts of the Janus biosensors have different surface modifications process. After the surface treatment, the two parts were assembled by the process which is shown in FIG. 11.

Finally, the morphology of the sensors with each of the surface modifications is shown in FIG. 12.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.
U.S. Patent App. Pub. No. 2019/037441
Baghel et al., Journal of Pharmaceutical Sciences, 105:2527-2544, 2016.
Czyrski and Sznura, Scientific Reports, 9:19458, 2019.
Damian et al., Journal of Optoelectronics and Advanced Materials, 9:780, 2007.
Davis et al., Journal of Pharmaceutical Sciences, S0022354920307413, 2020.
Fong et al., Expert Opinion on Drug Delivery, 14:403-426, 2017.
Handa et al., J Pharm Biomed Anal, 89:6-17, 2014.
Hayase et al. J Pharm Sci, 83:532-538, 1994.
Khuri and Mukhopadhyay, WIREs Computational Statistics. 2:128-149, 2010.
Lesaffre et al., Bayesian Methods in Pharmaceutical Research, CRC Press, 2020.
Majeed et al., Journal of Pharmacy and Pharmacology, 39:1044-1046, 1987.
Mäntele and Deniz, Spectrochimica Acta Part A: Molecular and Biomolecular Spectroscopy 173:965-968, 2017.
Matsuda et al., International Journal of Pharmaceutics, 54:211-221, 1989.
Noordin et al., Journal of Materials Processing Technology, 145:46-58, 2004.
Politis et al., Drug Development and Industrial Pharmacy, 43:889-901, 2017.
Sabir et al., Journal of Microencapsulation, 0:1-16, 2021.
Sadana & Ghogare, International Journal of Pharmaceutics, 70:195-199, 1991.
Thakkar et al., International Journal of Pharmaceutics, 576:118989, 2020.
Weissman and Anderson, Org. Process Res. Dev., 19:1605-1633, 2015.
Wichianphong and Charoenchaitrakool, Journal of CO2 Utilization, 26:212-220, 2018.
Zhang et al., International Journal of Pharmaceutics 119945, 2020.

What is claimed is:

1. A sensor system comprising:
(A) a plurality of a shapes, wherein the shape comprises a length, width, and height, wherein the height is sufficient to allow the distribution of recognition molecules along the height of the shape;
(B) one or more recognition molecules along with the height of the shape such that the recognition molecules are homogenously dispersed along the height of the shape; and
(C) a surface, wherein the plurality of shapes is affixed onto a surface, wherein the surface is configured to fit to enclose one or more wells on a commercial well plate wherein the shape is formed using an additive manufacturing technique.

2. The sensor system of claim 1, wherein the surface is configured to enclose all of the wells on the commercial well plate.

3. The sensor system of either claim 1 or claim 2, wherein the commercial well plate is a 96 well plate.

4. The sensor system of either claim 1 or claim 2, wherein the commercial well plate is 384 well plate.

5. The sensor system of either claim 1 or claim 2, wherein the commercial well plate is a specially designed well plate.

6. The sensor system according to any one of claims 1-5, wherein the shapes are prepared using a biopolymer.

7. The sensor system of claim 6, wherein the biopolymer is further defined as:
(i) a dimethacrylate monomer;
(ii) a first PEGylated methacrylate monomer, wherein the PEG segment comprises an average molecular weight from about 600 to about 1,000; and
(iii) a photoinitiator.

8. The sensor system of claim 7, wherein the biopolymer further comprises a second PEGylated methacrylate monomer, wherein the PEG segment comprises an average molecular weight from 100 to about 600.

9. The sensor system of either claim 7 or claim 8, wherein the biopolymer is used to prepare the shape and the surface.

10. The sensor system according to any one of claims 7-9, wherein the dimethacrylate monomer is joined by a linking group, wherein the linking group is an alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polymer of amino acid residues, a poly(ethylene glycol), a poly(propylene glycol), or a polycarbonate.

11. The sensor system of claim 10, wherein the linking group is alkanediyl_{(C≤12)}, substituted alkanediyl_{(C≤12)}, cycloalkanediyl_{(C≤12)}, substituted cycloalkanediyl_{(C≤12)}, heterocycloalkanediyl_{(C≤12)}, substituted heterocycloalkanediyl_{(C≤12)}, a poly(ethylene glycol), a poly(propylene glycol), or a polycarbonate.

12. The sensor system of either claim 10 or claim 11, wherein the linking group is alkanediyl_{(C≤12)} or substituted alkanediyl_{(C≤12)}.

13. The sensor system according to any one of claims 10-12, wherein the linking group is alkanediyl_{(C≤12)}.

14. The sensor system according to any one of claims 10-13, wherein the linking group is 2,2,4-trimethylhexyl or 2,4,4-trimethylhexyl.

15. The sensor system according to any one of claims 10-14, wherein the linking group further comprises two joining groups.

16. The sensor system of claim 15, wherein each of the joining groups is —O—, —NRₐ—, —C(O)—, —C(O)O—, —OC(O)—, —OC(O)O—, —C(O)NRₐ—, —NRₐC(O)—, —OC(O)NRₐ—, —NRₐC(O)O—, —S(O)ₐ—, —S(O)ₐO—, —OS(O)ₐ—, —OS(O)ₐO—, wherein: Rₐ is hydrogen, alkyl_{(C≤6)}, or substituted alkyl_{(C≤6)}; and a is 0, 1, or 2.

17. The sensor system of either claim 15 or claim 16, wherein each of the joining groups is —C(O)O—, —OC(O)—, —OC(O)O—, —C(O)NRₐ—, —NRₐC(O)—, —OC(O)NRₐ—, or —NRₐC(O)O—.

18. The sensor system according to any one of claims 15-17, wherein each of the joining groups is —OC(O)NRₐ— or —NRₐC(O)O—.

19. The sensor system according to any one of claims 15-18, wherein each of the joining group is —OC(O)NRₐ—.

20. The sensor system according to any one of claims 15-19, wherein each of the joining group is —OC(O)NH—.

21. The sensor system according to any one of claims 7-20, wherein the dimethacyrlate monomer is diurethane dimethacrylate.

22. The sensor system of claim 21, wherein the diurethane dimethacrylate is a mixture of multiple monomers.

23. The sensor system according to any one of claims 7-22, wherein the first PEGylated methacrylate monomer comprises an average molecular weight of the PEG unit from about 600 to about 800 Daltons.

24. The sensor system according to any one of claims 7-23, wherein the first PEGylated methacrylate monomer, wherein the average molecular weight of the PEG unit is about 700 Daltons.

25. The sensor system according to any one of claims 8-24, wherein the second PEGylated methacrylate monomer comprises an average molecular weight of the PEG unit from about 400 to about 600 Daltons.

26. The sensor system according to any one of claims 8-25, wherein the second PEGylated methacrylate monomer comprises an average molecular weight of the PEG unit from about 500 to about 600 Daltons.

27. The sensor system according to any one of claims 8-26, wherein the second PEGylated methacrylate monomer, wherein the average molecular weight of the PEG unit is about 575 Daltons.

28. The sensor system according to any one of claims 7-27, wherein the photoinitiator is a phosphorus based photoinitiator.

29. The sensor system according to any one of claims 7-28, wherein the photoinitiator is a phosphorus oxide based photoinitiator.

30. The sensor system according to any one of claims 7-29, wherein the photoinitiator is diphenyl(2,4,6-trimethylbenzoyl)phosphine oxide (TPO).

31. The sensor system according to any one of claims 7-30, wherein the biopolymer comprises from about 10 wt. % to about 65 wt. % of the dimethacrylate monomer in the pharmaceutical composition.

32. The sensor system according to any one of claims 7-31, wherein the biopolymer comprises from about 20 wt. % to about 40 wt. % of the dimethacrylate monomer in the biopolymer.

33. The sensor system according to any one of claims 7-32, wherein the biopolymer comprises from about 25 wt. % to about 35 wt. % of the dimethacrylate monomer in the pharmaceutical composition.

34. The sensor system according to any one of claims 7-33, wherein the biopolymer comprises from about 10 wt. % to about 55 wt. % of the first PEGylated methacrylate monomer in the biopolymer.

35. The sensor system according to any one of claims 7-34, wherein the biopolymer comprises from about 20 wt. % to about 40 wt. % of the first PEGylated methacrylate monomer in the biopolymer.

36. The sensor system according to any one of claims 7-35, wherein the biopolymer comprises from about 25 wt. % to about 35 wt. % of the first PEGylated methacrylate monomer in the biopolymer.

37. The sensor system according to any one of claims 8-36, wherein the biopolymer comprises from about 10 wt. % to about 55 wt. % of the second PEGylated methacrylate monomer in the biopolymer.

38. The sensor system according to any one of claims 8-37, wherein the biopolymer comprises from about 20 wt. % to about 40 wt. % of the second PEGylated methacrylate monomer in the biopolymer.

39. The sensor system according to any one of claims 8-38, wherein the biopolymer comprises from about 25 wt. % to about 35 wt. % of the second PEGylated methacrylate monomer in the biopolymer.

40. The sensor system according to any one of claims 7-39, wherein the biopolymer comprises from about 0.1 wt. % to about 5 wt. % of the photoinitiator in the biopolymer.

41. The sensor system according to any one of claims 7-40, wherein the biopolymer comprises from about 0.25 wt. % to about 3 wt. % of the photoinitiator in the biopolymer.

42. The sensor system according to any one of claims 7-41, wherein the biopolymer comprises from about 0.5 wt. % to about 2.5 wt. % of the photoinitiator in the biopolymer.

43. The sensor system according to any one of claims 7-42, wherein the biopolymer comprises a ratio of the dimethacrylate monomer to the first PEGylated methacrylate monomer from about 5:1 to about 1:5.

44. The sensor system according to any one of claims 7-43, wherein the biopolymer comprises a ratio of the dimethacrylate monomer to the first PEGylated methacrylate monomer from about 2:1 to about 1:2.

45. The sensor system according to any one of claims 7-44, wherein the biopolymer comprises a ratio of the dimethacrylate monomer to the first PEGylated methacrylate monomer is about 2:1, 1:1, or 2:3.

46. The sensor system according to any one of claims 8-45, wherein the biopolymer comprises a ratio of the dimethacrylate monomer to the second PEGylated methacrylate monomer from about 5:1 to about 1:5.

47. The sensor system according to any one of claims 8-46, wherein the biopolymer comprises a ratio of the dimethacrylate monomer to the second PEGylated methacrylate monomer from about 2:1 to about 1:2.

48. The sensor system according to any one of claims 8-47, wherein the biopolymer comprises a ratio of the dimethacrylate monomer to the second PEGylated methacrylate monomer is about 2:1, 1:1, or 2:3.

49. The sensor system according to any one of claims 8-48, wherein the biopolymer comprises a ratio of the first PEGylated methacrylate monomer to the second PEGylated methacrylate monomer from about 5:1 to about 1:5.

50. The sensor system according to any one of claims 8-49, wherein the biopolymer comprises a ratio of the first PEGylated methacrylate monomer to the second PEGylated methacrylate monomer from about 2:1 to about 1:2.

51. The sensor system according to any one of claims 8-50, wherein the biopolymer comprises a ratio of the first PEGylated methacrylate monomer to the second PEGylated methacrylate monomer is about 1:1 or 1:2.

52. The sensor system according to any one of claims 1-51, wherein the shape is a cylindrical shape.

53. The sensor system of claim 52, wherein the cylindrical shape comprises a height greater than its length or width.

54. The sensor system according to any one of claims 1-51, wherein the shape is a rectangular shape.

55. The sensor system of claim 54, wherein the rectangular shape comprises a height greater than its length or width.

56. The sensor system of either claim 54 or claim 55, wherein the rectangular shape comprises an equal length and width.

57. The sensor system according to any one of claims 1-51, wherein the shape is a ball shape.

58. The sensor system of claim 57, wherein the ball shape comprises a spherical end.

59. The sensor system of either claim 57 or claim 58, wherein the ball shape comprises a height longer than the width or length.

60. The sensor system according to any one of claims 57-59, wherein the ball shape comprise the spherical end on less than 50% of the height of the ball shape.

61. The sensor system according to any one of claims 1-51, wherein the shape comprises a first shape that is configured to bind to a second shape.

62. The sensor system of claim 61, wherein the first shape comprises a different recognition molecule than the second shape.

63. The sensor system according to any one of claims 1-51, wherein the shape is a custom shape for the application.

64. The sensor system according to any one of claims 1-51 and 63, wherein the shape is designed using CAD software.

65. The sensor system according to any one of claims 1-64, wherein the shape further comprises a biologic polymer.

66. The sensor system of claim 65, wherein the biologic polymer is a peptide, nucleic acid, or a polysaccharide.

67. The sensor system of claim 66, wherein the biologic polymer is a polysaccharide.

68. The sensor system according to any one of claims 65-67, wherein the biologic polymer comprises one or more amine groups.

69. The sensor system according to any one of claims 65-68, wherein the biologic polymer is a polysaccharide, wherein the sugar group comprises one or more amine groups.

70. The sensor system according to any one of claims 65-69, wherein the biologic polymer is chitosan.

71. The sensor system according to any one of claims 1-70, wherein the recognition molecule is covalently linked to the biologic polymer.

72. The sensor system according to any one of claims 1-71, wherein the covalent link between the biologic polymer and the recognition molecule is —Y₁-A₁-Y₂-A₂-Y₃—, wherein:
Y₁ is the biologic polymer;
Y₂ is alkanediyl_{(C≤12)}, alkenediyl_{(C≤12)}, arenediyl_{(C≤12)}, or a substituted version of any of these groups;
Y₃ is the recognition molecule; and
A₁ and A₂ are each independently selected from absent, —O—, —NRₐ—, —S—, —C(O)—, —CH(N—), —C(O)O—, —C(O)NH—, —OC(O)O—, —OC(O)NH—, —NHC(O)NH—, —C(NRₐ)O—, —C(NRₐ)NH—, —OC(NRₐ)O—, —OC(NRₐ)NH—, —NHC(NRₐ)NH—;
wherein:
Rₐ is hydrogen, alkyl_{(C≤6)}, or substituted alkyl_{(C≤6)}.

73. The sensor system of claim 72, wherein Y₂ is alkanediyl_{(C≤12)} or substituted alkanediyl_{(C≤12)}.

74. The sensor system of either claim 72 or claim 73, wherein Y₂ is alkanediyl_{(C≤12)}.

75. The sensor system according to any one of claims 72-74, wherein Y₂ is ethylene, propylene, or butylene.

76. The sensor system according to any one of claims 72-75, wherein Y₂ is propylene.

77. The sensor system according to any one of claims 72-76, wherein A₁ is —CH(N—).

78. The sensor system according to any one of claims 72-77, wherein A₂ is —CH(N—).

79. The sensor system according to any one of claims 72-78, wherein A₁ and A₂ are —CH(N—).

80. The sensor system according to any one of claims 1-79, wherein the recognition molecule is a biologic molecule.

81. The sensor system of claim 80, wherein the biologic molecule is an antibody, a sugar or polysaccharide, an amino acid or a polypeptide, or a nucleic acid or polynucleotide.

82. The sensor system of either claim 80 or claim 81, wherein the biologic molecule is a polypeptide.

83. The sensor system of claim 82, wherein the polypeptide is a protein.

84. The sensor system of claim 83, wherein the protein is streptavidin.

85. The sensor system of either claim 80 or claim 81, wherein the biologic molecule is a nucleic acid.

86. The sensor system of claim 85, wherein the nucleic acid is an aptamer.

87. The sensor system of either claim 80 or claim 81, wherein the biological molecule is an antibody.

88. The sensor system according to any one of claims 1-79, wherein the recognition molecule is a small molecule.

89. The sensor system according to any one of claims 1-88, wherein the recognition molecule further comprises a second recognition molecule.

90. The sensor system of claim 89, wherein the second recognition molecule is an antibody, a signaling molecule, a nucleic acid or polynucleotide, an amino acid or a polypeptide, a sugar or polysaccharide, or a small molecule.

91. The sensor system of either claim 89 or claim 90, wherein the second recognition molecule is a small molecule.

92. The sensor system according to any one of claims 89-91, wherein the second recognition molecule is biotin.

93. The sensor system according to any one of claims 89-92, wherein the second recognition molecule is bound to a signaling molecule.

94. The sensor system of claim 93, wherein the signaling molecule is a dye.

95. The sensor system of either claim 93 or claim 94, wherein the signaling molecule is a fluorophore.

96. The sensor system according to any one of claims 1-95, wherein the recognition molecule binds to a macromolecule.

97. The sensor system of claim 96, wherein the macromolecule is a biologic molecule.

98. The sensor system of claim 97, wherein the biologic molecule is an antibody, a sugar or polysaccharide, an amino acid or a polypeptide, or a nucleic acid or polynucleotide.

99. The sensor system according to any one of claims 1-95, wherein the recognition molecule binds to a small molecule.

100. The sensor system of claim 99, wherein the small molecule is an active pharmaceutical ingredient, a co-factor, a vitamin, or a hormone.

101. The sensor system of claim 100, wherein the active pharmaceutical ingredient is a metabolite of the active pharmaceutical ingredient.

102. The sensor system according to any one of claims 1-101, wherein the sensor system comprises a detection limit of less than 1 μmol per well.

103. The sensor system of claim 102, wherein the detection limit is less than 100 nmol per well.

104. The sensor system of either claim 102 or claim 103, wherein the detection limit is less than 10 nmol per well.

105. The sensor system according to any one of claims 102-104, wherein the detection limit is less than 1 nmol per well.

106. The sensor system according to any one of claims 102-105, wherein the detection limit is less than 100 pmol per well.

107. The sensor system according to any one of claims 102-106, wherein the detection limit is less than 10 pmol per well.

108. The sensor system according to any one of claims 102-107, wherein the detection limit is less than 1 pmol per well.

109. The sensor system according to any one of claims 1-108, wherein the sensor system has a detection limit from about 0.5 pmols to about 2 μmols per well for a 96 well plate.

110. The sensor system according to any one of claims 1-108, wherein the sensor system has a detection limit from about 0.25 pmols to about 1 μmols per well for a 384 well plate.

111. A method of detecting the presence of an analyte in a solution comprising reacting a solution containing the analyte to a sensor system according to any one of claims 1-110.

112. The method of claim 111, wherein the method further comprises reacting the sensor system with a detection modality.

113. The method of claim 112, wherein the detection modality is a dye labeled antibody.

114. The method of claim 113, wherein the dye is a fluorophore.

115. The method of claim 111, wherein the method further comprises allowing the analyte to displace a detection modality from the sensor system; and washing the sensor system to remove the detection modality.

116. The method according to any one of claims 111-115, wherein the solution further comprises a buffer.

117. The method of claim 116, wherein the buffer maintains the pH of the solution at from about 4 to about 10.

118. The method of claim 117, wherein the pH of the solution is from about 6 to about 9.

119. The method of either claim 117 or claim 118, wherein the pH of the solution is from about 7 to about 8.

120. The method according to any one of claims 117-119, wherein the pH of the solution is about 7.5.

121. The method according to any one of claims 116-120, wherein the buffer is a sulfonic acid-based buffer.

122. The method of claim 121, wherein the sulfonic acid-based buffer is HEPES or HEPBS.

123. The method according to any one of claims 116-122, wherein the buffer is HEPBS.

124. The method according to any one of claims 116-123, wherein the solution comprises a concentration of the buffer from about 0.1 mM to about 250 mM.

125. The method of claim 124, wherein the concentration of the buffer is from about 1 mM to about 100 mM.

126. The method of either claim 124 or claim 125, wherein the concentration of the buffer is about 20 mM.

127. The method according to any one of claims 111-126, wherein the solution further comprises a salt.

128. The method of claim 127, wherein the salt is a sodium salt.

129. The method of either claim 127 or claim 128, wherein the salt is sodium chloride.

130. The method according to any one of claims 127-129, wherein the solution comprises a concentration of the salt from about 1 mM to about 25 M.

131. The method of claim 130, wherein the concentration of the salt is from about 10 mM to about 10 M.

132. The method of either claim 130 or claim 131, wherein the concentration of the salt is about 1 M.

133. The method according to any one of claims 111-132, wherein the solution further comprises a surfactant.

134. The method of claim 133, wherein the surfactant is a non-ionic surfactant.

135. The method of either claim 133 or claim 134, wherein the surfactant is Tween.

136. The method according to any one of claims 133-135, wherein the surfactant is Tween 20.

137. The method according to any one of claims 133-136, wherein the solution comprises an amount of the surfactant from about 0.0001% to about 1%.

138. The method of claim 137, wherein the concentration of the surfactant is from about 0.001% to about 0.1%.

139. The method of either claim 137 or claim 138, wherein the concentration of the surfactant is about 0.01%.

140. The method according to any one of claims 111-139, wherein the solution further comprises a protein.

141. The method of claim 140, wherein the protein is a serum albumin.

142. The method of claim 141, wherein the serum albumin is bovine serum albumin.

143. The method according to any one of claims 111-142, wherein the solution further comprises a reducing agent.

144. The method of claim 143, wherein the reducing agent is a borohydride based reducing agent.

145. The method of either claim 143 or claim 144, wherein the reducing agent is sodium cyanoborohydride.

146. The method according to any one of claims 143-145, wherein the solution comprises a concentration of the reducing agent from about 1 mM to about 1 M.

147. The method of claim 146, wherein the concentration of reducing agent is from about 10 mM to about 500 mM.

148. The method of either claim 146 or claim 147, wherein the concentration of reducing agent is from about 50 mM to about 100 mM.

149. A method of determining the presence of absence of a disease state in a patient comprising exposing a sample from the patient to the sensor system according to any one of claims 1-110.

150. The method of claim 149, wherein the method determines the presence of a disease state.

151. The method of claim 149, wherein the method determines the absence of a disease state.

152. The method according to any one of claims 149-151, wherein the patient is a mammal.

153. The method of claim 152, wherein the mammal is human.

154. The method according to any one of claims 149-153, wherein the sample is saliva, blood, urine, mucous, plasma, serum, sputum, stool, or tears.
